(19) United States
(12) Patent Application Publication
(10) Pub. No.: US 2011/0275569 A1
(43) Pub. Date: Nov. 10, 2011

(54) PROTEASES TARGETING AGENTS

(75) Inventors: Mosé ROSSI, Arcofelice (NA) (IT); Gianna PALMIERI, Pozzuoli (NA) (IT); Giuliana CATARA, NAPOLI (IT); Ruvo MENOTTI, Trevico (AV) (IT)

(73) Assignee: CONSIGLIO NAZIONALE DELLE RICERCHE, Rome (IT)

(21) Appl. No.: 13/093,199

(22) Filed: Apr. 25, 2011

Related U.S. Application Data
(63) Continuation-in-part of application No. PCT/IT2009/000478, filed on Oct. 23, 2009.

(30) Foreign Application Priority Data
Oct. 23, 2008 (IT) NA2008A000060

(51) Int. Cl. A61K 38/16; C12N 5/10; C12N 1/19; C12N 1/21; C12N 9/99; C12P 19/34; C07K 14/00; C07H 21/04; C07H 21/02; C07K 7/06; C12P 21/02; C07K 7/08; A61K 31/7088; A61K 38/10; A61P 35/00; A61P 25/00; A61P 9/00; A61P 7/00; A61P 13/00; A61P 29/00; A61P 11/00; C12N 15/63

(52) U.S. Cl. 514/13.5; 435/320.1; 435/325; 435/419; 435/254.2; 435/348; 435/358; 435/365; 435/367; 435/370; 435/352; 435/252.31; 435/252.33; 435/184; 435/91.2; 435/366; 530/350; 536/23.7; 536/24.5; 530/328; 435/69.1; 530/326; 530/324; 514/20.1; 514/44 R; 514/21.4; 514/19.3; 514/17.7; 514/16.4

(57) ABSTRACT

A protein inhibitor from Sulfolobulus Solfataricus and peptides thereof, which both have the ability to inhibit AARE and elastase. Similarly to the eukaryotic counterparts, the recombinant protein is able to inhibit in vitro the bovine alfa-chymotrypsin with a high specificity, and the porcine elastase but not all commercial available trypsins, features which distinguish all the members belonging to the family PEBP. Through site-specific mutagenesis techniques of the gene codifying SsCEI, it has been recognized the “reactive site loop”—RCL—on the inhibitor, responsible for the interaction with the eukarial protease target already identified. The inhibitor and the peptides thereof can be used as new compounds capable of modulating cognitive enhancement cardiovascular diseases, cancer, inflammation, hematological diseases, neurological diseases and urological diseases.

### FIELD OF INVENTION

The invention relates to cognitive enhancement, cardiovascular diseases, cancer, inflammation, hematological diseases, neurological diseases, urological diseases and therapeutic modulation thereof. In particular, the invention relates to compounds based on peptides and derivatives capable of modulating cognitive enhancement cardiovascular diseases, cancer, inflammation, hematological diseases, neurological diseases and urological diseases.

### BACKGROUND OF THE INVENTION

### Endogenous Protease Inhibitors.

Protease inhibitors are divided into different classes that are widely distributed in all three domains of life, but so far considerable attention has been dedicated exclusively to those from eukaryotes, where the number of genes codifying for these proteins varies from 10 up to hundreds for each family. Moreover, the genome comparative analysis of the different microorganisms available in the network, has revealed that only three families of protease inhibitors are present in Archaea in common with prokaryotes and eukaryotes, the “serpins” (clan I4), “chagasins” (clan I42) (Rawlings et al. 2004) and the “phosphatidylethanolamine binding proteins” (PEBP, I51 clans). In particular, the family I51 constitutes a distinct “cluster”, whose members do not have any region homologous to the other protease inhibitors studied so far. The founder of the family I51 is the protein of the yeast TFS1 able to inhibit the carboxypeptidase Y with an inhibition constant of 1.8 nM, while in rats, the pebp3 inhibits thrombin, the neuropsin and the chymotrypsin (Hengst et al. 2001) with an inhibition constant in the micro-molar range. The PEBPs represent a family of multifunctional proteins, involved in a number of physiological and cellular processes of the body, as the spermatogenesis, the control of heart activity, cerebral development in mammals, or inflorescence in plants (Serre et al. 1998). Proteins belonging to this family show unique structural characteristics. Despite highly conserved conformations, they have significant structural differences, especially on their N and C-terminal ends, which could provide the discriminatory properties for the various biological functions, such as the modulation of their interaction with proteins or ligands The biological activity of the PEBPs may be divided into two broad categories: i) they are able to interact and therefore to regulate the activity of various enzymes and proteins, such as serin proteases, kinases (Yeung et al. 1999) and transcription factors in the cells; ii) they show the capacity to specifically interact with many anionic ligands such as phospholipids, nucleotides and derivatives of cholesterol and hormones. Despite the wide dissemination of PEBPs in tissue and cellular compartments of many species, little is known about the several functions they perform. The cellular localization and the binding with phospholipids on the inner surface of plasma membrane has suggested for some members of this family a role in the organization of plasma membrane, during growth, development and differentiation of cells. Furthermore, in the central nervous system some serine proteases are able to block the morphological changes of neuronal cells associated with the activity of proteolytic enzymes, as shown in rats by PEBP. These findings suggest a role of this class of inhibitors in the development and control of neuronal processes (Hengst et al. 2001). Therefore, there is today a strong request to better define the biological roles of this class of proteins in humans. For example, changes in the expression of PEBPs are related to many pathological conditions that affect a significant percentage of the population, for which the current therapeutic strategies are often ineffective (primary and metastatic cancer, diabetes, Alzheimer's disease, sterility, (Chen et al. 2006). It is therefore clear that these proteins are a starting point for medical research. Currently, the pebp-1 human is recognized in the US as a prognostic factor for assessing the risk of spread of prostate cancer cells in the body.

### Proteases

Proteases play a central role in controlled processes, such as blood coagulation, fibrinolysis, complement activation, fertilization, and hormone production. These enzymes are also used in a variety of diagnostic, therapeutic, and industrial contexts. [Jones et al. (1991), Proc. Nat. Acad. Sci. 88: 2194-2198; WO 02/068579].

Proteases belong systematically to the C—N Hydrolases. More specifically, proteases catalyze the hydrolytic cleavage of a peptide bond and are therefore called peptidases as well.

Peptidases located extracellularly in the blood or other extracellular compartments of the body play often regulatory roles in processes like for example blood clotting, fibrinolysis, or activation of complement constituents.

Intracellularly located proteases also exhibit a wide variety of roles. They are found in compartments like the ER, the Golgi apparatus, or the lysosomes. Their functions include for example activation of peptide hormons, ubiquitin mediated proteolysis, among others. Proteases are most commonly classified according to their mechanism of action, or to specific active groups that are present in the so-called reactive center.

### Serine Peptidases

Serine proteases exhibit a serine in the catalytic site which forms a covalent ester intermediate during the catalytic reaction pathway by a nucleophilic attack on the carboxy carbon of the peptide bond. In the active site of serine proteases, a catalytic triad comprised of an aspartate, a histidine and the above mentioned serine is found. This triad functions in the reaction mechanism as a charge relay system.

To the large family of serine proteases belong, for example, the digestive enzymes trypsin and chymotrypsin, components of the complement cascade, enzymes involved in the blood clotting cascade, as well as enzymes that function in degradation, rebuilding and maintenance of constituents of the extracellular matrix.

Elastase and Acyl-aminoacyl-peptidase (AARE or APH or ACPH or APEH) are also members of the group of serine protease enzymes.

### Elastase

Human neutrophil elastase (elastase) is believed to be a main responsible for inflammatory conditions in the lung, where an imbalance between elastase and antiproteases leads to proteolytic destruction of lung elastin and tissue (Snider G L. Emphysema: the first two centuries and beyond. Am Rev Respir Dis 1992; 146:1334-44, 1615-22). Elastase is a serine protease of the chymotrypsin family that is stored in the primary (azurophil) granules of polymorphonuclear neutrophils (PMNs). It was first identified as degradative enzymes responsible for eliminating intracellular pathogens and breaking down tissues at inflammatory sites [A. Janoff, J. Scherer, Mediators of inflammation in leukocyte lysosomes. IX. Elastinolytic activity in granules of human polymorphonuclear leukocytes, J. Exp. Med. 128 (1968) 1137e1155. R. C. Kao, N. G. Wehner, K. M. Skubitz, B. H. Gray, J. R. Hoidal, Proteinase 3. A distinct human polymorphonuclear leukocyte proteinase that produces emphysema in hamsters, J. Clin. Invest. 82 (1988) 1963e1973. M. Baggiolini, J. Schnyder, U. Bretz, B. Dewald, W. Ruch, Cellular mechanisms of proteinase release from inflammatory cells and the degradation of extracellular proteins, Ciba Found. Symp. (1979) 105-121], and was soon recognized as a possible molecular target for anti-inflammatory agents [G. D. Virca, G. Metz, H. P. Schnebli, Similarities between human and rat leukocyte elastase and cathepsin G, Eur. J. Biochem. 144 (1984) 1-9.]. The imbalance between human neutrophil elastase and endogenous serine proteinase inhibitors is considered to cause a variety of elastase-mediated inflammatory disorders, including emphysema, which is due to a1-antitrypsin deficiency, and cystic fibrosis (CF) [Bettina Siedle, Andrea Hrenn, Irmgard Merfort, Natural Compounds as Inhibitors of Human Neutrophil Elastase, Planta Med., 2007; 73: 401-420, Georg Thieme Verlag KG Stuttgart]. Thus, elastase has been the object of intensive research to find potent inhibitors that target its destructive and pro-inflammatory action. A large body of work has been so far carried out on alpha-1 antitrypsin [Camelier A A, Winter D H, Jardim J R, Barboza C E, Cukier A, Miravitlles, “Alpha-1 antitrypsin deficiency: diagnosis and treatment” J Bras Pneumol. 2008 July; 34(7):514-27], a serpin which is the natural inhibitor of elastase. The block of a1-antitrypsin processing in hepatocytes significantly reduces levels of circulating a1-antitrypsin, which may lead to emphysema due to insufficient protection of the lower respiratory tract from elastase, permitting progressive destruction of the alveoli. In CF, impaired mucocilliary clearance leads to chronic bacterial infections and subsequent vigorous influx of neutrophils in the airways. High levels of elastase are released and induce progressive proteolytic impairment of multiple defense pathways leading to endobronchial obstruction and airway wall destruction. Studies also indicate that elastase contributes to chronic inflammatory airway diseases by inducing mucin production in airway epithelial cells. Elastase is also often associated with ARDS, although observational studies of humans do not yet convincingly demonstrate the role for elastase.

### AARE or APEH

AARE catalyses the hydrolysis of the amino-terminal peptide bond of an N-acetylated protein to generate a N-acetylated amino acid and a protein with a free amino-terminus. NH2 terminal acetylation of intracellular proteins occurs in many eukaryotic cells and more rarely in prokaryotic cells. About 40% of these acetylated proteins are structural proteins, consisting mostly of histones, virus coat proteins, keratins, actins, ribosomal proteins, crystallins, myelin proteins, tropomyosin.

The acetylamino acid formed by acylpeptide hydrolase is further processed to acetate and a free amino acid by an aminoacylase. The substrates for the acylpeptide hydrolase and the acylase behave in a reciprocal manner since acylpeptide hydrolase binds but does not process acetylamino acids and the acylase binds acetylpeptides but does not hydrolyze them; however, the 2 enzymes share the same specificity for the acyl group. All of these findings indicate common functional features in the protein structures of the 2 enzymes, which are encoded by the same region of human chromosome 3, namely, 3p21. [Jones et al. (1991), Proc. Nat. Acad. Sci. 88: 2194-2198] suggested that there may be a relationship between the expression of these 2 enzymes and acetylated peptide growth factors in some carcinomas.

As a huge number of proteases play a central role in several important cellular and intracellular processes, their value as pharmaceutical targets has been proven for several of these enzymes. Therefore, the identification of specific inhibitors or modulators may lead to the development of novel compounds useful in pharmacological approaches to treat diseases and conditions in which protease activities are involved. In the specific case of elastase and AARE, these diseases may include, but are not limited to cardiovascular diseases, cancer, inflammation, hematological diseases, neurological diseases and urological diseases in a mammal (see Patent Application WO/2007/147496).

The invention relates to pharmaceutical compositions for the treatment of cardiovascular diseases, cancer, inflammation, hematological diseases, neurological diseases and urological diseases in a mammals, which are due to an altered activity of AARE and elastase.

In particular, a protein inhibitor from Sulfolobulus Solfataricus and peptides thereof, which both have the ability to inhibit AARE and elastase are here described as the object of the invention. The inhibitor has been obtained in recombinant form and purified to homogeneity from the hyperthermophile Sulfolobus solfataricus. It is a monomer of molecular mass of 19.0 kDa that shows a high degree of similarity with the sequences of rat PEBP1 (28% identity), human PEBP1 (27% identity), TFS1 yeast (33% identity) and E. coli YBCL (38% identity). Similarly to the eukaryotic counterparts, the recombinant protein is able to inhibit in vitro the bovine alfa-chymotrypsin with a high specificity, and the porcine elastase but not all commercial available trypsins, features which distinguish all the members belonging to the family PEBP. Moreover, through site-specific mutagenesis techniques of the gene codifying SsCEI, it has been recognized the “reactive site loop”—RCL—on the inhibitor, responsible for the interaction with the eukarial protease target already identified. Such a site shows an aminoacid sequence never found in any protease inhibitor of chymotrypsin-like enzymes, so far characterized.

On the basis of the inhibitor RCL, isolated peptides of different length, sequence and structure, can be designed and tested in vitro for their capacity to block or enhance (modulate) the activity of the target proteases. These peptides, where provided of the suitable properties of stability, potency and selectivity, can be used as new compounds for the treatment of diseases whereby the proteases are involved.

### SUMMARY OF INVENTION

According to a first aspect of the invention there is provided a protein encoded by the following nucleotide sequence (SEQ. ID No 1):

| ORIGIN | |
|---|---|
| ttgaatagtg aaagtatata ttcgatgaga gtagtatctt cagcctttaa gaatgaggat | 60 |
| tttataccta ttaaatatac ttgtgatgga caagatctgt ctccggagtt agagtgggat | 120 |
| cttgttacta acgctaaaag ttatgcgata atcgtagaag atccagatgc gcctggagga | 180 |
| actttcatac attgggtaat atacaacata actaccaata gattgccaga aggagtgcca | 240 |
| agactgtaca aatcacaata tggtgtacaa ggtgtaaacg attttgggaa tatagggtat | 300 |
| aacggtccgt gtcctcctaa gacacatcca cctcatagat attattttta tgtttatgca | 360 |
| attgatacaa tactacttga aattaaaaat attaatgctg ataagttaaa atcactaatg | 420 |
| gagggacatg ggatagagag aggattcgta atgggtaaat ataagagaaa ataa | |

which is translated into the following protein sequence (SEQ ID No 2):

MNSESIYSMRVVSSAFKNEDFIPIKYTCDGQDLSPELEWDLVTNAKSYAI
IVEDPDAPGGTFIHWVIYNITTNRLPEGVPRLYKSQYGVQGVNDFGNIGY
NGPCPPKTHPPHRYYFYVYAIDTILLEIKNINADKLKSLMEGHGIERGFV
MGKYKRK.

This protein is one of 1323 cytosolic proteins identified from the organism and expressed in the middle exponential phase of growth (Chong, et al, 2005), although no functional role has yet been assigned. The S. solfataricus inhibitor has been isolated following its ability to inhibit the activity of bovine alpha-chymotrypsin. The protein is monomeric with an isoelectric point of 6.7 and a molecular mass of 19.0 kDa.

On the basis of the inhibitor RCL, isolated peptides of different length, sequence and structure, have been designed and tested in vitro for their capacity to block the activity of the target protease. These peptides do block the activity of AARE, therefore, according to a second aspect of the invention there is provided a compound of formula I:

I: Y1-X1-X2-X3-X4-X5-X6-X7-X8-X9-X10-X11-X12-X13-
X14-X15-X16-Y2

wherein,
X1 is the L- or D-enantiomer of Tyrosine or null;
X2 is the L- or D-enantiomer of Alanine or null when X1 is null;
X3 is the L- or D-enantiomer of Isoleucine or null when X1 and X2 are null;
X4 is the L- or D-enantiomer of Aspartic acid or null when X1 through X3 are null;
X5 is the L- or D-enantiomer of Threonine or null when X1 through X4 are null;
X6 is the L- or D-enantiomer of Isoleucine, or D-enantiomer of Aspartic acid, or D-enantiomer of Cysteine with an acetamidomethyl (Acm) protection group on the sulphidryl group (abbreviated as Cys(Acm), or D-enantiomer of Proline.
X7 is the L- or D-enantiomer of Leucine or of Alanine, or the D-enantiomer of Methionine, or D-enantiomer of Cys(Acm), or D-enantiomer of Aspartic acid.
X8 is the L- or D-enantiomer of Leucine or of Alanine, or D-enantiomer of Methionine, or D-enantiomer of Cys(Acm).
X9 is the L- or D-enantiomer of Glutamic acid, or D-enantiomer of Aspartic acid or D-enantiomer of Cys(Acm), or D-enantiomer of Arginine, or D-enantiomer of Histidine.
X10 is the L- or D-enantiomer of Isoleucine or null when X11 through X16 are null;
X11 is the L- or D-enantiomer of Lysine or null when X12 through X16 are null;
X12 is the L- or D-enantiomer of Asparagine or null when X13 through X16 are null;
X13 is the L- or D-enantiomer of Isoleucine or null when X14 through X16 are null;
X14 is the L- or D-enantiomer of Asparagine or null when X15 and X16 are null;
X15 is the L- or D-enantiomer of Alanine or null when X16 is null;
   and
X16 is the L- or D-enantiomer of Aspartic acid or null.

The “null” notation, indicates that a number of other peptide sequences can be generated by the formula I with the indicated combination of L- and D-aminoacids, by progressively shortening the sequence from the N- and/or the C-termini until at least tetrapeptides of formula:

Y1-(D or L)-Ile-(D or L)-Leu(or Ala)-(D or L)-Leu-
(D or L)-Glu-Y2

are generated.

In this formula:
Ile is the aminoacid Isoleucine;
Leu is the aminoacid Leucine;
Ala is the aminoacid Alanaine;
Glu is the aminoacid glutamic acid.

All the retro variants corresponding to each single sequence generated from the general formula I, according to Fischer P M, Curr Protein Pept Sci. 2003 October; 4(5):339-56, “The design, synthesis and application of stereochemical and directional peptide isomers: a critical review”, represent a further embodiment to this invention. The retro variant of a generic peptide is intended a new molecule whereby any carbonyl group within the peptide backbone is exchanged with an NH group and viceversa and where the C-terminal carboxylic group and the N-terminal amino group are interconverted.

An amide bond is intended to occur between each single aminoacid of the sequences, as it is well known to those skilled in peptide and protein chemistry. Likewise, in the general formula reported above, Y1 and Y2 represent chemical groups, forming amide bonds, added to the N-terminal amino-group or to the C-terminal carbonyl of the peptide.

Following this notation, when Y1 is an hydrogen, a free amine is generated at the N-terminus; when Y1 is an acetyl group, an N-terminal acetylated peptide is generated. Alternatively Y1 can be one of the following chemical groups which can help to improve the half-life of resulting molecules or improve their delivery properties by increasing cell permeability:
any linear carboxylic acid having from 3 up to 20 carbon atoms
any PEG derivative having a carbonyl group so as to form an amide bond with the N-terminal amine.

Y1 can also be an acetyl group, whenever the first aminoacid on the N-terminus is in its D configuration. Y2 is an hydroxyl group so as to generate a terminal carboxylic acid or is one of the following groups added to the carbonyl acid terminal of the oligopeptide sequence so as to form an amide bond:
an amine group to form an amide or
any linear primary amine having from 1 up to 20 carbon atoms or
any PEG derivative having an amine group so as to form an amide bond with the C-.terminal carboxylic group.

In a further embodiment of this invention, derivatives of compound of formula I are included; said derivatives are selected from the group consisting of:
a) oligomers or multimers of molecules of the compound of formula I, said oligomers and multimers comprising two or more molecules of the compound of formula I each linked to a common scaffold moiety via an amide bond formed between an amine or carboxylic acid group present in molecules of the compound of formula I and an opposite amine or carboxylic acid group on a scaffold moiety said scaffold moiety participating in at least 2 amide bonds,
b) derivatives comprising a molecule of the compound of formula I or an oligomer or multimer as defined above in part a) conjugated via an ester bond, an ether bond or a thioether bond, on the N or C terminal amino- or carboxylic acid group of the peptide moiety to:
   PEG,
   PEG-based compounds,
   fatty acids,
and
c) salts and solvates of a molecule of the compound of formula I or of a derivative as defined in part a) or b) above.

In a further embodiment of this invention, peptides generated by the general formula I and carrying at least one methyl group on an amide nitrogen (N-methyl-aminoacids) are disclosed. Such derivatives, reportedly have an increased capacity to pass cell membranes and also the BBB (Blood Brain Barrier). See reference [Malakoutikhah M, Teixidó M, Giralt E, “Toward an optimal blood-brain barrier shuttle by synthesis and evaluation of peptide libraries”. J Med. Chem. 2008 Aug. 28; 51(16):4881-91.

According to a third aspect of the invention, there is provided a pharmaceutical composition comprising a compound according to the first and the second aspect of the invention and a pharmaceutically acceptable carrier.

According to the first and the second aspect of the invention, there is provided a method of treating cardiovascular diseases, cancer, inflammation, hematological diseases, neurological diseases and urological diseases, comprising administering a compound according to the first and the second aspect of the invention or, according to the third aspect of the invention, a pharmaceutical composition comprising a compound according to the first and the second aspect of the invention to a subject in need thereof.

According to a fourth aspect of the invention, there is provided a compound according to the first and second aspect of the invention or a composition according to the third aspect of the invention for use as a medicament.

According to a fifth aspect of the invention, there is provided use of a compound according to the first and second aspects of the invention or a pharmaceutical composition according to the third aspect of the invention for the manufacture of a medicament for the treatment of cardiovascular diseases, cancer, inflammation, hematological diseases, neurological diseases and urological diseases.

### BRIEF DESCRIPTION OF THE DRAWINGS

FIG. 1 shows the alignment of the amino acid sequence of the inhibitor with that of a homologous protein, the YBCL from E. coli (1FUX), a member of “151” family.

FIG. 2 shows the binding of Sso0767 to alpha-chymotrypsin.

FIG. 3 shows the inhibition kinetics analysis.

FIG. 4 shows the binding of Sso0767 to alpha-chymotrypsin.

FIG. 5 shows inhibition activity of the SsCEI 3 versus AARE 0.2 μg.

FIG. 6 shows the aminoacid sequence of SsCEI protein.

FIG. 7 shows inhibition of porcine acylamino-acid-releasing enzyme (AARE) by inhibitor SsCEI 1-4.

FIG. 8A shows markedly reduced proteasomal activity of SsCEI 4.

FIG. 8B shows markedly reduced proteasomal activity of SsCEI 2.

FIG. 8C shows caspase-3 activity.

### DETAILED DESCRIPTION OF THE INVENTION

The strategy underlining the present invention arises from an understanding that modulating (blocking, reducing or increasing) the activity of the proteases AARE and elastase is a way to control a number of diseases including cognitive enhancement, cardiovascular diseases, cancer, inflammation, hematological diseases, neurological diseases and urological diseases. In particular, recent evidences have pointed out that blocking the activity of AARE can enhance the cognitive abilities of individuals with neurodegenerative or neurovegetative disorders [Olmos C, Sandoval R, Rozas C, Navarro S, Wyneken U, Zeise M, Morales B, Pancetti F. “Effect of short-term exposure to dichlorvos on synaptic plasticity of rat hippocampal slices: involvement of acylpeptide hydrolase and alpha(7) nicotinic receptors”. Toxicol Appl Pharmacol. 2009 Jul. 1; 238(1):37-46], whereas, enhancing its activity would be a way to reduce symptoms associated with cardiovascular diseases, cancer, inflammation, hematological diseases, neurological diseases and urological diseases. Also blocking or reducing the activity of elastase is a widely accepted approach for controlling inflammation in lung diseases such as emphysema.

To this end, the inventors have identified a protein derived from Sulfolobulus solfataricus and several peptides thereof isolated from the Reactive Center Loop (RCL) [Palmieri G, Catara G, Saviano M, Langella E, Gogliettino M, Rossi M. J “First Archaeal PEPB-Serine Protease Inhibitor from Sulfolobus solfataricus with Noncanonical Amino Acid Sequence in the Reactive-Site Loop”, Proteome Res. 2009 January; 8(1):327-34], possessing the ability to modulate the activity of such proteases. Peptides with inhibitory activity of AARE and of elastase have been identified by biochemical assays and have inhibitory activity in the range of the micromolar concentration.

According to the convention, all peptides presented herein are described from the N terminus to the C terminus. According to certain preferred embodiments, the compounds are essentially short peptides having from 4 up to 16 aminoacids with optional blocking groups Y1 and Y2 at one or more of the termini.

### Derivatives

Preferably, derivatives of the compound of the first and second aspect of the invention are functional derivatives. The term “functional derivative” is used herein to denote a chemical derivative of the protein or of the compound of formula I having the same physiological function as the corresponding unmodified counterpart or, alternatively, having the same in vitro function in a functional assay (for example, in one of the assays described in one of the examples disclosed herein).

### Polyethylene Glycol (PEG)

The invention encompasses PEGylated derivates. For ease in handling and manufacturing, the preferred molecular weight of a polyethylene glycol for derivatisation of a compound of the invention is from about 1 kDa to about 100 kDa, the term “about” indicating that in preparations of polyethylene glycol, some molecules will weigh more, some less, than the stated molecular weight. Polymers of other molecular weights may be used, depending on the desired therapeutic profile, for example the duration of sustained release desired, the effects, if any on biological activity, the ease in handling, the degree or lack of antigenicity and other known effects of the polyethylene glycol to a therapeutic protein or analog. For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa.

Preferably, derivatives of the protein or of the peptides in formula I, retain at least 50% of its capacity to modulate the activity of AARE and elastase, compared to the underivatized counterparts, as assessed in an in vitro inhibition assay.

According to certain preferred embodiments, the oligopeptide core moiety of the compound, identified as X1 trough X16 in Formula I, has an amino acid sequence selected from the group consisting of:

| | |
|---|---|
| SEQ ID No. 3: | (L-Ile)-(L-Leu)-(L-Leu)-(L-Glu), |
| SEQ ID No. 4: | (L-Ile)-(L-Leu)-(L-Ala)-(L-Glu), |
| SEQ ID No. 5: | (D-Ile)-(D-Leu)-(D-Leu)-(D-Glu), |
| SEQ ID No. 6: | (D-Ile)-(D-Leu)-(D-Ala)-(D-Glu), |
| SEQ ID No. 7: | (D-Ile)-(L-Leu)-(L-Leu)-(L-Glu), |
| SEQ ID No. 8: | (L-Ile)-(D-Leu)-(L-Leu)-(L-Glu), |
| SEQ ID No. 9: | (L-Ile)-(L-Leu)-(D-Leu)-(L-Glu), |
| SEQ ID No. 10: | (L-Ile)-(L-Leu)-(L-Leu)-(D-Glu), |
| SEQ ID No. 11: | (D-Ile)-(L-Leu)-(L-Ala)-(L-Glu), |
| SEQ ID No. 12: | (L-Ile)-(D-Leu)-(L-Ala)-(L-Glu), |
| SEQ ID No. 13: | (L-Ile)-(L-Leu)-(D-Ala)-(L-Glu), |
| SEQ ID No. 14: | (L-Ile)-(L-Leu)-(L-Ala)-(D-Glu), |
| SEQ ID No. 15: | (D-Ile)-(D-Leu)-(L-Leu)-(L-Glu), |
| SEQ ID No. 16: | (L-Ile)-(D-Leu)-(D-Leu)-(L-Glu), |
| SEQ ID No. 17: | (L-Ile)-(L-Leu)-(D-Leu)-(D-Glu), |
| SEQ ID No. 18: | (D-Ile)-(L-Leu)-(L-Leu)-(D-Glu), |
| SEQ ID No. 19: | (L-Ile)-(D-Leu)-(D-Leu)-(L-Glu), |
| SEQ ID No. 20: | (D-Ile)-(L-Leu)-(D-Leu)-(L-Glu), |
| SEQ ID No. 21: | (L-Ile)-(D-Leu)-(L-Leu)-(D-Glu), |
| SEQ ID No. 22: | (D-Ile)-(D-Leu)-(L-Ala)-(L-Glu), |
| SEQ ID No. 23: | (L-Ile)-(D-Leu)-(D-Ala)-(L-Glu), |
| SEQ ID No. 24: | (L-Ile)-(L-Leu)-(D-Ala)-(D-Glu), |
| SEQ ID No. 25: | (D-Ile)-(L-Leu)-(L-Ala)-(D-Glu), |
| SEQ ID No. 26: | (L-Ile)-(D-Leu)-(D-Ala)-(L-Glu), |
| SEQ ID No. 27: | (D-Ile)-(L-Leu)-(D-Ala)-(L-Glu), |
| SEQ ID No. 28: | (L-Ile)-(D-Leu)-(L-Ala)-(D-Glu), |
| SEQ ID No. 29: | (L-Glu)-(L-Leu)-(L-Leu)-(L-Ile), |
| SEQ ID No. 30: | (L-Glu)-(L-Ala)-(L-Leu)-(L-Ile), |
| SEQ ID No. 31: | (D-Glu)-(L-Leu)-(L-Leu)-(L-Ile), |
| SEQ ID No. 32: | (L-Glu)-(D-Leu)-(L-Leu)-(L-Ile), |
| SEQ ID No. 33: | (L-Glu)-(L-Leu)-(D-Leu)-(L-Ile), |
| SEQ ID No. 34: | (L-Glu)-(L-Leu)-(L-Leu)-(D-Ile), |
| SEQ ID No. 35: | (D-Glu)-(L-Ala)-(L-Leu)-(L-Ile), |
| SEQ ID No. 36: | (L-Glu)-(D-Ala)-(L-Leu)-(L-Ile), |
| SEQ ID No. 37: | (L-Glu)-(L-Ala)-(D-Leu)-(L-Ile), |
| SEQ ID No. 38: | (L-Glu)-(L-Ala)-(L-Leu)-(D-Ile), |
| SEQ ID No. 39: | (D-Glu)-(D-Leu)-(L-Leu)-(L-Ile), |
| SEQ ID No. 40: | (L-Glu)-(D-Leu)-(D-Leu)-(L-Ile), |
| SEQ ID No. 41: | (L-Glu)-(L-Leu)-(D-Leu)-(D-Ile), |
| SEQ ID No. 42: | (D-Glu)-(L-Leu)-(L-Leu)-(D-Ile), |
| SEQ ID No. 43: | (L-Glu)-(D-Leu)-(D-Leu)-(L-Ile), |
| SEQ ID No. 44: | (D-Glu)-(L-Leu)-(D-Leu)-(L-Ile), |
| SEQ ID No. 45: | (L-Glu)-(D-Leu)-(L-Leu)-(D-Ile), |
| SEQ ID No. 46: | (D-Glu)-(D-Ala)-(L-Leu)-(L-Ile), |
| SEQ ID No. 47: | (L-Glu)-(D-Ala)-(D-Leu)-(L-Ile), |
| SEQ ID No. 48: | (L-Glu)-(L-Ala)-(D-Leu)-(D-Ile), |
| SEQ ID No. 49: | (D-Glu)-(L-Ala)-(L-Leu)-(D-Ile), |
| SEQ ID No. 50: | (L-Glu)-(D-Ala)-(D-Leu)-(L-Ile), |
| SEQ ID No. 51: | (D-Glu)-(L-Ala)-(D-Leu)-(L-Ile), |
| SEQ ID No. 52: | (L-Glu)-(D-Ala)-(L-Leu)-(D-Ile), |

### Oligomers and Multimers

The second aspect of the invention encompasses, oligomers or multimers of molecules of the compound of formula I, said oligomers and multimers comprising 2 or more molecules of the compound of formula I each linked to a common scaffold moiety via an amide bond formed between an amine or carboxylic acid group present in molecules of the compound of formula I and an opposite amino or carboxylic acid group on a scaffold moiety said scaffold moiety participating in at least 2 amide bonds as it is reported in Tam, J. P. (1988) Proc Natl Acad Sci USA 85(15), 5409-5413.

According to certain embodiments the common scaffold may be the amino acid lysine. Lysine is a tri-functional amino acid, having in addition to the functional groups which define it as an amino acid, a amino group on its side claim. This tri-functional nature allows it to form up to three amide bonds with peptides or similar molecules. Other tri-functional standard amino acids which may be used as a common scaffold include α,β-diaminopropionic acid (Dap), α,δ-diaminobutirricacid (Dab), Ornitine. Other tri-functional non-standard amino acids may also be used in accordance with the invention. The common scaffold may also comprise branched peptides which incorporate tri-functional amino acids within their sequence and have at least three functionally active terminal groups able to form amide bonds.

### Fatty Acids

Fatty acid derivatives of a compound of the invention comprising a compound of formula I, linked to a fatty acid via a disulfide linkage may be used for delivery of a compound of the invention to cells and tissues. Lipidisation markedly increases the absorption of the compounds relative to the rate of absorption of the corresponding unlipidised compounds, as well as prolonging blood and tissue retention of the compounds. Moreover, the disulfide linkage in lipidised derivative is relatively labile in the cells and thus facilitates intracellular release of the molecule from the fatty acid moieties. Suitable lipid-containing moieties are hydrophobic substituents with 4 to 26 carbon atoms, preferably 5 to 19 carbon atoms. Suitable lipid groups include, but are not limited to, the following: palmityl (C15H31,), oleyl (C15H29), stearyl (C17H35), cholate; linolate, and deoxycholate.

### Salts and Solvates

Salts and solvates of compounds of the invention that are suitable for use in a medicament are those wherein a counterion or associated solvent is pharmaceutically acceptable. However, salts and solvates having non-pharmaceutically acceptable counterions or associated solvents are within the scope of the present invention, for example, for use as intermediates in the preparation of the compounds of formula (I) and their pharmaceutically acceptable salts or solvates.

Suitable salts according to the invention include those formed with organic or inorganic acids or bases. Pharmaceutically acceptable acid addition salts include those formed with hydrochloric, hydrobromic, sulphuric, nitric, citric, tartaric, acetic, phosphoric, lactic, pyruvic, acetic, trifluoroacetic, succinic, perchloric, fumaric, maleic, glycollic, lactic, salicylic, oxaloacetic, methanesulfonic, ethanesulfonic, p-toluenesulfonic, formic, benzoic, malonic, naphthalene-2-sulfonic, benzenesulfonic, and isethionic acids. Other acids such as oxalic, while not in themselves pharmaceutically acceptable, may be useful as intermediates in obtaining the compounds of the invention and their pharmaceutical acceptable salts. Pharmaceutically acceptable salts with bases include ammonium salts, alkali metal salts, for example potassium and sodium salts, alkaline earth metal salts, for example calcium and magnesium salts, and salts with organic bases, for example dicyclohexylamine and N-methyl-D-glucosamine.

Those skilled in the art of organic chemistry will appreciate that many organic compounds can form complexes with solvents in which they are reacted or from which they are precipitated or crystallized. Such complexes are known as “solvates”. For example, a complex with water is known as a “hydrate”. The present invention provides solvates of compounds of the invention.

Examples of preferred molecules of formula I include:

L-normal peptides
(SEQ ID No. 53)

| | |
|---|---|
| 1: | H-(L)Tyr-(L)Ala-(L)Ile-(L)Asp-(L)-Thr-(L)-Ile- |
| | (L)Leu-(L)Leu-(L)Glu)-(L)Ile-(L)Lys-(L)Asn- |
| | (L)Ile-(L)Asn-(L)Ala-(L)Asp-NH2 |

(SEQ ID No. 54)

| | |
|---|---|
| 2: | H-(L)Tyr-(L)Ala-(L)Ile-(L)Asp-(L)-Thr-(L)-Ile- |
| | (L)Leu-(L)Ala-(L)Glu)-(L)Ile-(L)Lys-(L)Asn- |
| | (L)Ile-(L)Asn-(L)Ala-(L)Asp-NH2 |

(SEQ ID No. 55)

| | |
|---|---|
| 3: | H-(L)Thr-(L)-Ile-(L)Leu-(L)Leu-(L)Glu)-(L)Ile- |
| | (L)Lys-(L)Asn-(L)Ile-(L)Asn-(L)Ala-(L)Asp-NH2 |

(SEQ ID No. 56)

| | |
|---|---|
| 4: | H-(L)-Thr-(L)-Ile-(L)Leu-(L)Ala-(L)Glu)- |
| | (L)Ile-(L)Lys-(L)Asn-(L)Ile-(L)Asn-(L)Ala- |
| | (L)Asp-NH2 |

D-normal peptides
(SEQ ID No. 57)

| | |
|---|---|
| 5: | H-(D)Tyr-(D)Ala-(D)Ile-(D)Asp-(D)-Thr-(D)-Ile- |
| | (D)Leu-(D)Leu-(D)Glu)-(D)Ile-(D)Lys-(D)Asn- |
| | (D)Ile-(D)Asn-(D)Ala-(D)Asp-NH2 |

(SEQ ID No. 58)

| | |
|---|---|
| 6: | H-(D)Tyr-(D)Ala-(D)Ile-(D)Asp-(D)-Thr-(D)-Ile- |
| | (D)Leu-(D)Ala-(D)Glu)-(D)Ile-(D)Lys-(D)Asn- |
| | (L)Ile-(D)Asn-(D)Ala-(D)Asp-NH2 |

(SEQ ID No. 59)

| | |
|---|---|
| 7: | H-(D)Thr-(D)-Ile-(D)Leu-(D)Leu-(D)Glu)-(D)Ile- |
| | (D)Lys-(D)Asn-(D)Ile-(D)Asn-(D)Ala-(D)Asp-NH2 |

(SEQ ID No. 60)

| | |
|---|---|
| 8: | H-(D)-Thr-(D)-Ile-(D)Leu-(D)Ala-(D)Glu)- |
| | (D)Ile-(D)Lys-(D)Asn-(D)Ile-(D)Asn-(D)Ala- |
| | (D)Asp-NH2 |

D-retro peptides
(SEQ ID No. 61)

| | |
|---|---|
| 9: | H-(D)Asp-(D)Ala-(D)Asn-(D)Ile-(D)Asn-(D)Lys- |
| | (D)Ile-(D)Glu-(D)Leu-(D)Leu-(D)Ile-(D)Thr- |
| | (D)Asp-(D)Ile-(D)Ala-(D)Tyr-NH2 |

(SEQ ID No. 62)

| | |
|---|---|
| 10: | H-(D)Asp-(D)Ala-(D)Asn-(D)Ile-(D)Asn-(D)Lys- |
| | (D)Ile-(D)Glu-(D)Ala-(D)Leu-(D)Ile-(D)Thr- |
| | (D)Asp-(D)Ile-(D)Ala-(D)Tyr-NH2 |

(SEQ ID No. 63)

| | |
|---|---|
| 11: | H-(D)Asp-(D)Ala-(D)Asn-(D)Ile-(D)Asn-(D)Lys- |
| | (D)Ile-(D)Glu-(D)Leu-(D)Leu-(D)Ile-(D)Thr-NH2 |

(SEQ ID No. 64)

| | |
|---|---|
| 12: | H-(D)Asp-(D)Ala-(D)Asn-(D)Ile-(D)Asn-(D)Lys- |
| | (D)Ile-(D)Glu-(D)Ala-(D)Leu-(D)Ile-(D)Thr-NH2 |

### Pharmaceutical Compositions

According to a third aspect of the invention, there is provided a pharmaceutical composition comprising a compound according to the first and second aspect of the invention and a pharmaceutically acceptable carrier.

While it is possible for the active ingredient to be administered alone, it is preferable for it to be present in a pharmaceutical formulation or composition. Accordingly, the invention provides a pharmaceutical formulation comprising a compound of formula (I) or derivatives thereof, or a salt or solvate thereof, as defined above and a pharmaceutically acceptable carrier. Pharmaceutical compositions of the invention may take the form of a pharmaceutical formulation as described below.

The pharmaceutical formulations according to the invention include those suitable for oral, parenteral (including subcutaneous, intradermal, intramuscular, intravenous, and intraarticular), inhalation (including fine particle dusts or mists which may be generated by means of various types of metered does pressurized aerosols, nebulizers or insufflators), rectal and topical (including dermal, transdermal, transmucosal, buccal, sublingual, and intraocular) administration, although the most suitable route may depend upon, for example, the condition and disorder of the recipient. The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste. Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2 S, 1988.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The present compounds can, for example, be administered in a form suitable for immediate release or extended release. Immediate release or extended release can be achieved by the use of suitable pharmaceutical compositions comprising the present compounds, or, particularly in the case of extended release, by the use of devices such as subcutaneous implants or osmotic pumps. The present compounds can also be administered liposomally. Preferably, compositions according to the invention are suitable for subcutaneous administration, for example by injection.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate and/or lactose and/or other excipients, binders, extenders, disintegrants, diluents and lubricants such as those known in the art. The compounds of formula I or variant, derivative, salt or solvate thereof can also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor. An aqueous carrier may be, for example, an isotonic buffer solution at a pH of from about 3.0 to about 8.0, preferably at a pH of from about 3.5 to about 7.4, for example from 3.5 to 6.0, for example from 3.5 to about 5.0. Useful buffers include sodium citrate-citric acid and sodium phosphate-phosphoric acid, and sodium acetate/acetic acid buffers. The composition preferably does not include oxidizing agents and other compounds that are known to be deleterious to the compound of formula I and related molecules. Excipients that can be included are, for instance, other proteins, such as human serum albumin or plasma preparations. If desired, the pharmaceutical composition may also contain minor amounts of non-toxic auxiliary substances, such as wetting or emulsifying agents, preservatives, and pH buffering agents and the like, for example sodium acetate or sorbitan monolaurate.

Exemplary compositions for nasal aerosol or inhalation administration include solutions in saline, which can contain, for example, benzyl alcohol or other suitable preservatives, absorption promoters to enhance bioavailability, and/or other solubilizing or dispersing agents such as those known in the art. Conveniently in compositions for nasal aerosol or inhalation administration the compound of the invention is delivered in the form of an aerosol spray presentation from a pressurized pack or a nebulizer, with the use of a suitable propellant, e.g., dichlorodifluoro-methane, trichlorofluoromethane, dichlorotetrafluoroethane, carbon dioxide or other suitable gas.—In the case of a pressurized aerosol the dosage unit can be determined by providing a valve to deliver a metered amount. Capsules and cartridges of e.g., gelatin for use in an inhaler or insufflator can be formulated to contain a powder mix of the compound and a suitable powder base, for example lactose or starch. In one specific, non-limiting example, a compound of the invention is administered as an aerosol from a metered dose valve, through an aerosol adapter also known as an actuator. Optionally, a stabilizer is also included, and/or porous particles for deep lung delivery are included (e.g., see U.S. Pat. No. 6,447,743).

Formulations for rectal administration may be presented as a retention enema or a suppository with the usual carriers such as cocoa butter, synthetic glyceride esters or polyethylene glycol. Such carriers are typically solid at ordinary temperatures, but liquefy and/or dissolve in the rectal cavity to release the drug.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase (mineral oil gelled with polyethylene).

Preferred unit dosage formulations are those containing an effective dose, as hereinbefore recited, or an appropriate fraction thereof, of the active ingredient. It should be understood that in addition to the ingredients particularly mentioned above, the formulations of this invention may include other agents conventional in the art having regard to the type of formulation in question, for example those suitable for oral administration may include flavouring agents.

The compounds of the invention are also suitably administered as sustained-release systems. Suitable examples of sustained-release systems of the invention include suitable polymeric materials, for example semi-permeable polymer matrices in the form of shaped articles, e.g., films, or microcapsules; suitable hydrophobic materials, for example as an emulsion in an acceptable oil; or ion exchange resins; and sparingly soluble derivatives of the compound of the invention, for example, a sparingly soluble salt. Sustained-release systems may be administered orally; rectally; parenterally; intracistemally; intravaginally; intraperitoneally; topically, for example as a powder, ointment, gel, drop or transdermal patch; bucally; or as an oral or nasal spray.

Preparations for administration can be suitably formulated to give controlled release of compounds of the invention. For example, the pharmaceutical compositions may be in the form of particles comprising one or more of biodegradable polymers, polysaccharide jellifying and/or bioadhesive polymers, amphiphilic polymers, agents capable of modifying the interface properties of the particles of the compound of formula (I). These compositions exhibit certain biocompatibility features which allow a controlled release of the active substance. See U.S. Pat. No. 5,700,486.

A compound of the invention may be delivered by way of a pump (see Langer, supra; Sefton, CRC Crit. Ref. Biomed. Eng. 14:201, 1987; Buchwald et al., Surgery 88:507, 1980; Saudek et al., N. Engl. J. Med. 321:574, 1989) or by a continuous subcutaneous infusions, for example, using a mini-pump. An intravenous bag solution may also be employed. Other controlled release systems are discussed in the review by Langer (Science 249:1527-1533, 1990). In another aspect of the disclosure, compounds of the invention are delivered by way of an implanted pump, described, for example, in U.S. Pat. No. 6,436,091; U.S. Pat. No. 5,939,380; U.S. Pat. No. 5,993,414.

Implantable drug infusion devices are used to provide patients with a constant and long term dosage or infusion of a drug or any other therapeutic agent. Essentially such device may be categorized as either active or passive. A compound of the present invention may be formulated as a depot preparation. Such a long acting depot formulation can be administered by implantation, for example subcutaneously or intramuscularly; or by intramuscular injection. Thus, for example, the compounds can be formulated with suitable polymeric or hydrophobic materials, for example as an emulsion in an acceptable oil; or ion exchange resins; or as a sparingly soluble derivatives, for example, as a sparingly soluble salt.

A therapeutically effective amount of a compound of the invention may be administered as a single pulse dose, as a bolus dose, or as pulse doses administered over time. Thus, in pulse doses, a bolus administration of a compound of the invention is provided, followed by a time period wherein no a compound of the invention is administered to the subject, followed by a second bolus administration. In specific, non-limiting examples, pulse doses of a compound of the invention are administered during the course of a day, during the course of a week, or during the course of a month.

In one embodiment, a therapeutically effective amount of a compound of the invention is administered with a therapeutically effective amount of another agent, for example a further anti-neoplastic chemotherapeutic agent (for example, thalidomide, dexamethasone, bortezomib, and lenalidomide) or an agent to treat anaemia (for example erythropoietin), or an agent to prevent bone fractures (for example a bisphosphonate such as pamidronate or zoledronic acid).

According to the fourth aspect of the invention, there is provided a method of treating cardiovascular diseases, cancer, inflammation, hematological diseases, neurological diseases and urological diseases, comprising administering a therapeutically effective amount of a compound according to the first and second aspect of the invention or a pharmaceutical composition according to the third aspect of the invention to a subject in need thereof.

According to a fifth aspect of the invention, there is provided a compound according to the first and second aspect of the invention or a composition according to the third aspect of the invention for use as a medicament.

According to a sixth aspect of the invention, there is provided use of a compound according to the first and second aspect of the invention or a pharmaceutical composition according to the third aspect of the invention for the manufacture of a medicament for the treatment of cardiovascular diseases, cancer, inflammation, hematological diseases, neurological diseases and urological diseases.—Said diseases and subject being defined in certain preferred embodiments as described above in reference to the fourth aspect of the invention.

### EXAMPLES

The following non-limiting examples illustrate the invention.

### Example 1

### Synthesis of Compounds SsCEI 1, SsCEI 2, SsCEI 3, SsCEI1 4

By way of example, synthesis of Compounds I to 4 is reported. Compounds SsCEI 1 to 4, of SEQ ID No. 68, 69, 70, 71, respectively as reported in Table IV and V, comprise L-normal peptides with a free N-terminus and a protected C-terminus by means of an amide bond.

Peptide SsCEI 1 to 4 were prepared following the Fmoc/tBu solid phase method (Fields G. B. and Noble R. L. 1990 Int J Pept Protein Res; 35: 161-214; Bodansky, M. and Bodansky A. 1995). The practice of peptide synthesis, 2nd edn., Springer Verlag, Berlin) using a SYRO multiple peptide synthesizer (Multisynthech, Germany). The synthesis scale was 50 μmoles. 100 mg of RINK amide polystyrene resin (Fmoc-RINK-AM-resin, GL Biochem, Shangai, China), having a substitution of 0.50 mmoles/g was used for all the synthesis. The resin was placed in 5 mL polypropylene vessels endowed with a 20 μm teflon septum. At the beginning the resin was swollen with 3.0 mL of a 50:50 dichloromethane (DCM):dimethyl formamide (DMF) mixture (both from LabScan, Stillorgan, Ireland) for 20 minutes. The 4 reaction vessels were placed in the synthesizer and a protocol with in situ preactivation steps by using a 0.5 M solution of Benzotriazole-1-yl-oxy-tris-pyrrolidino-phosphonium hexafluorophosphate (PyBOP, Novabiochem) in DCM, and pure di-iso-propyl-ethylamine (DIEA, Sigma-Aldrich) was adopted to attach the first aminoacids and to elongate the chain. The aminoacids, in their Fmoc-protected form were also employed at 0.5 M in DMF (Fields G. B. and Noble R. L. 1990 Int J Pept Protein Res; 35: 161-214). The acylation time was 30 minutes, at room temperature (RT). The Fmoc deprotection was achieved by a solution of DMF-Piperidine 6:4 mixture (Piperidine, Pip, Sigma-Aldrich, Milan, Italy) for 20 minutes at RT. The reactante were removed under vacuum and the resins washed 3 times with 1.5 mL of DMF after each cycle of coupling and deprotection. After synthesis completion, the resins were extensively washed with DMF, DCM, Methyl Alcohol (MeOH, LabScan), and Ethyl Alcohol (Et20, LabScan), and dried under vacuum. To cleave the peptides from the resins, they were treated with 3.0 mL/each with a mixture composed of TFA-H2O-TIS 90:5:5 (v/v/v) mixture (TFA, Trifluoroacetic acid, Sigma-Aldrich; TIS, tri-iso-propylsilane, Sigma-Aldrich) for 3 hours at RT. Resins were removed by filtration, then 20 mL of cold Et2O was added to the trifluoroacetic solution, leading to the formation of a white precipitate. After removal of the solvents by centrifugation, the precipitates were washed with 10.0 mL of cold Et2O, dissolved in 5.0 mL of H2O/CH3CN 50:50 (v/v) and lyophilized. Peptides were characterized by LC-MS using a narrow bore 30×2 mm ID ONYX C18 column (Phenomenex, Torrance, Calif., USA), equilibrated at 600 μL/min with 5% CH3CN, 0.05% TFA. The analysis was carried out applying a gradient of CH3CN, 0.05% TFA from 5% to 60% over 3 minutes. The peptide was purified by semi-preparative RP-HPLC using a 10×1 cm C18 ONYX column (Phenomenex, Torrance, Calif., USA), equilibrated at 20 mL/min, injecting 10 mg in each run. A gradient from 5% to 65% over 8 minutes was applied to elute the peptides. Pure fractions were pooled and characterized by LC-MS. The determined MW of Peptides 1 to 4 are reported in the following Table I. Products more than 95% pure (by HPLC) were obtained. A typical yield of about 40% was achieved after purification of all the crude products.

TABLE I

| Sequence | | Expected | Exper. | Purity |
|---|---|---|---|---|
| number | compound | MW (amu) | MW (amu) | (%) |
| 1 | SsCEI 1 | 1818.12 | 1818.5 | >95 |
| 2 | SsCEI 2 | 1355.61 | 1355.4 | >95 |
| 3 | SsCEI 3 | 1776.04 | 1776.2 | >95 |
| 4 | SsCEI 4 | 1313.53 | 1313.6 | >95 |

### Example 2

### Identification and Isolation of the Inhibitor Sso0767

A computational analysis of the S. solfataricus genome revealed the presence of a single gene (sso0767) encoding a putative protein inhibitor belonging to the PEBP family I51. This protein is one of 1323 cytosolic proteins identified from the organism and expressed in the middle exponential phase of growth (Chong, et al, 2005), although no functional role has yet been assigned. The S. solfataricus inhibitor, SsCEI, was isolated following its ability to inhibit the activity of bovine alpha-chymotrypsin.

### Example 3

### Isolation, Cloning and Recombinant Expression in E. coli of the Gene Sso0767

Gene sso0767 (reported hereinafter as SEQUENCE 1) was obtained by PCR amplification directly from total genomic DNA of S. solfataricus P2, using the following oligonucleotides, of SEQ ID No 65 and 66, respectively:

(SEQ ID No. 65)
P1 5′-CCATGGGCTTGAATAGTGAAAGTATATA-3′
and
(SEQ ID No. 66)
P2 5′-GAGCTCTTTTCTCTTATATTTACCCATTACGAAT-3′.

The NcoI and XhoI restriction sites, underlined, were introduced to allow the insertion of the gene in expression vector. The experiments by PCR amplification (Hybaid) were performed under standard conditions and PCR product was cloned into the vector pMOS (Promega) and subcloned into expression vector pET-28 (Novagen). Cells of E. coli BL21—CodonPlus RIL (Stratagene) containing the plasmid pET28-0767 were grown at 37° C. in the middle LB (50 ml) containing kanamycin (0.05 mg mL-1) until reaching an OD600 of 0.6. Protein expression was induced by the addition of 1 mM IPTG followed by 4 h growth.

Nucleotidic sequence:
SEQ. ID N. 1
sso0767
ttgaatagtg aaagtatata ttcgatgaga gtagtatctt
cagcctttaa gaatgaggat tttataccta ttaaatatac
ttgtgatgga caagatctgt ctccggagtt agagtgggat
cttgttacta acgctaaaag ttatgcgata atcgtagaag
atccagatgc gcctggagga actttcatac attgggtaat
atacaacata actaccaata gattgccaga aggagtgcca
agactgtaca aatcacaata tggtgtacaa ggtgtaaacg
attttgggaa tatagggtat aacggtccgt gtcctcctaa
gacacatcca cctcatagat attattttta tgtttatgca
attgatacaa tactacttga aattaaaaat attaatgctg
ataagttaaa atcactaatg gagggacatg ggatagagag
aggattcgta atgggtaaat ataagagaaa ataa
Aminoacid sequence:
SEQ. ID N. 2
SsCEI:
MNSESIYSMRVVSSAFKNEDFIPIKYTCDGQDLSPELEWDLVTNAKSYAI
IVEDPDAPGGTFIHWVIYNITTNRLPEGVPRLYKSQYGVQGVNDFGNIGY
NGPCPPKTHPPHRYYFYVYAIDTILLEIKNINADKLKSLMEGHGIERGFV
MGKYKRK

### Example 4

### Purification and Characterization of the Recombinant S. solfataricus Inhibitor Sso0767 (SsCEI)

The E. coli recombinant cells were suspended in 0.05 M sodium phosphate buffer and broken with a French press. The supernatant was heated for 30 min at 70° C. and after centrifugation the inhibitor, ‘HIS-tagged’ to the C-terminus, was purified using a HIS-Select Spin column (Sigma). The further purification of recombinant inhibitor was obtained by molecular exclusion chromatography on Superdex G75 column. The homogeneity of the protein has been demonstrated by SDS-PAGE analysis, Edman degradation and molecular exclusion chromatography. The inhibitor was purified as a monomer with an isoelectric point of 6.7 and a molecular mass of 19.0 kDa as determined by SDS-PAGE analysis, molecular exclusion chromatography and electrospray mass spectrometry. We refer to the sequence of the isolated gene as SEQ ID N. 1 and to the deduced amino acid sequence as SEQ ID N. 2 (157 amino acids). Analysis of sequence similarity by utilizing different database searching revealed that the Sso0767 (or SsCEI) is a member of the family I51 of protease inhibitors, showing all the structural features typical of PEBP proteins (phosphoethanolamine-binding protein), which are the members this inhibitor cluster (Hengst U, et al 2001; Mima et al 2003).

The purified inhibitor shows high resistance to the common protein denaturants and is resistant to several digestive proteases. Furthermore, the inhibitor is highly thermostable, in fact no reduction in inhibitory activity was observed even after 4 days of protein incubation at 90° C.

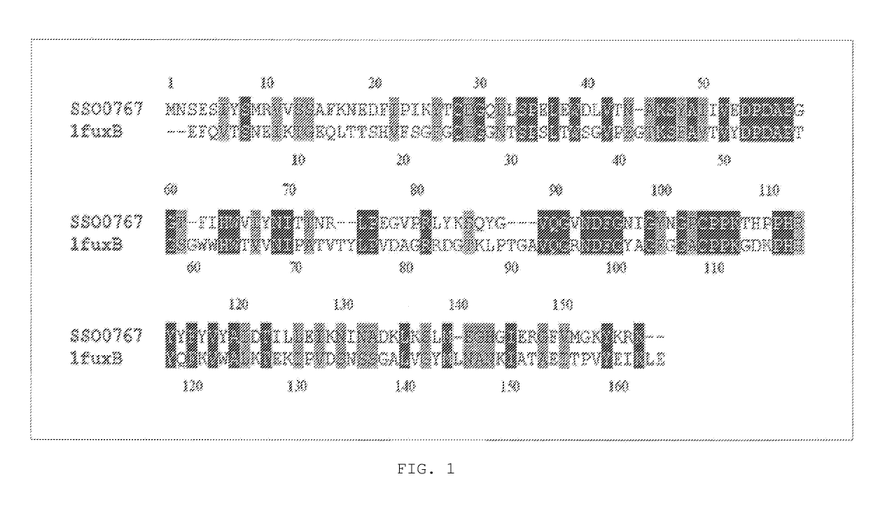

FIG. 1 shows the alignment of the amino acid sequence of the inhibitor with that of a homologous protein, the YBCL from E. coli (1FUX), a member of “I51” family. Regions highlighted in black are the conserved regions among SsCEI and the analyzed protein, while those in gray correspond to segments with accepted amino acid substitution. Gaps are denoted by dashes.

The 3D structure of the E. coli protein (Serre et al 2001) was used as template to build a very reliable 3D model of the S. solfataricus Sso0767. Based on this model it was assumed that the inhibitor, as evidenced in YBCL, contains a disulfide bridge linking the cysteine in position 29 and 114.

### Example 5

### Substrate Specificity, Kinetics, and Analysis of Sso1273 Reactive Site Loop

To identify the biological function of the purified inhibitor Sso0767 (SsCEI), the ability to inhibit a number of proteases was tested. The set of the proteases examined includes: the alpha-chymotrypsin, trypsin, elastase, carboxypeptidase Y, subtilisin, thrombin and two thermophilic proteases, the kumamolisin-ac and pernisine. Beside inhibition activity toward α-chymotrypsin which is the best target protease among those tested, results showed a clear activity versus elastase, while trypsin, carboxypeptidase Y, subtilisin or thrombin were not affected by Sso1273. The specific interaction between the inhibitor and the target proteases was also examined by incubating Sso1273 in a reaction mixture containing all the proteases investigated. In these conditions, the catalytic performance of the inhibitor was identical to that observed by assaying each protease alone.

FIG. 2 shows the binding of Sso0767 to alpha-chymotrypsin.

The inhibition of Sso1273 (SsCEI) versus α-chymotrypsin followed a hyperbolic pattern with increasing concentrations of the inhibitor and the IC50 value (50% inhibitory concentration) was 0.10 μM (Table II). Since the secondary plot (the slope of inhibition graph versus Sso1273 concentration) was linear, it was suggested that the application of Michaelis-Menten inhibition kinetics was appropriate in this study. The inhibition constant Ki, determined by the double reciprocal plot was 0.08 μM, revealing a higher affinity of Sso1273 for α-chymotrypsin with respect to the cognate PEBP serine protease inhibitors.

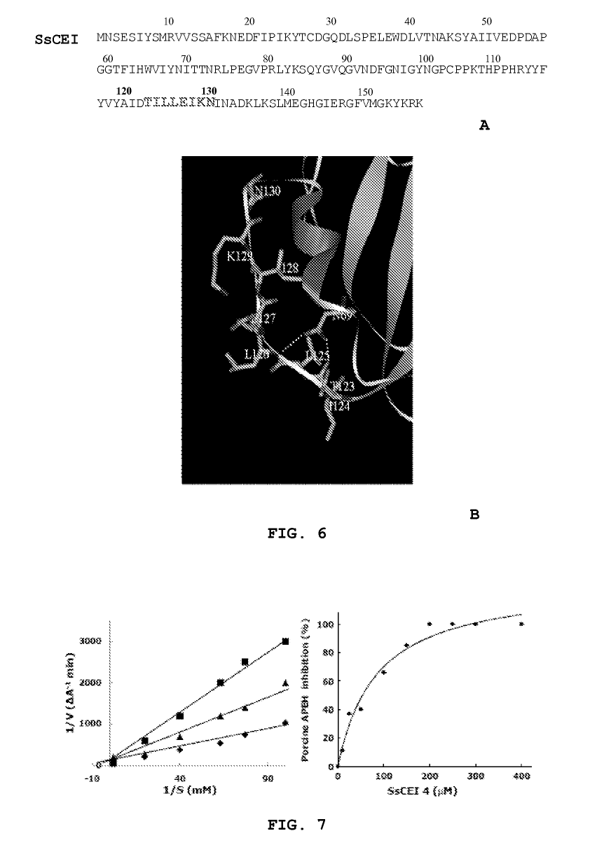

FIG. 6 shows the aminoacid sequence of SsCEI protein. In A is illustrated the sequence of the inhibitor protein from S. solfataricus, SsCEI; the sequence of the active site (RSL) is underlined. In B a model detail of RSL in SsCEI is shown, active residues are labeled.

A more detailed inspection of the SsCEI model and of the structural requirements occurring in chymotrypsin/elastase specific inhibitors, allowed to propose that the reactive site loop (RSL) of the inhibitor encloses the sequence encompassing from residue 123 to residue 130: TILLEIKN (SEQ ID No. 67), in which the scissile peptide bond is L126 (P1)-E127 (P′1). This sequence is located on an external, protruding loop located possessing an extended conformation similar to other reactive sites in serine protease inhibitors. The functional properties of the inhibitor active site (RSL) responsible of the specific inhibition activity towards porcine and human APEH, were strongly validated by mutagenesis analysis.

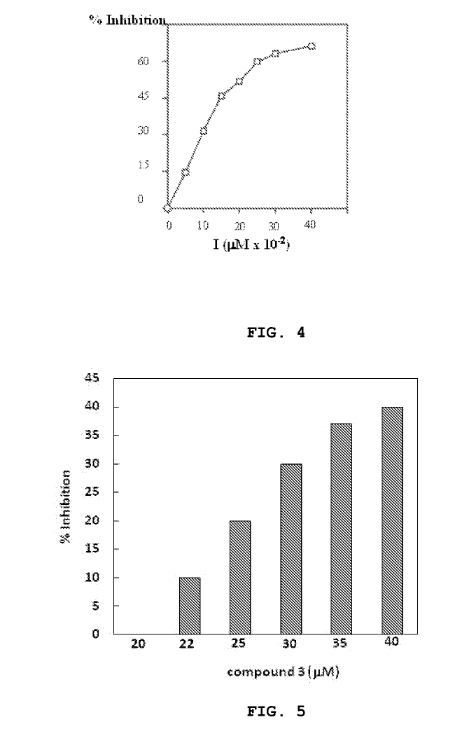

FIG. 4 shows the binding of Sso0767 to alpha-chymotrypsin.

The inhibition of Sso1273 (SsCEI) versus α-chymotrypsin followed a hyperbolic pattern with increasing concentrations of the inhibitor and the IC50 value (50% inhibitory concentration) was 0.10 μM (Table II). Since the secondary plot (the slope of inhibition graph versus Sso1273 concentration) was linear, it was suggested that the application of Michaelis-Menten inhibition kinetics was appropriate in this study. The inhibition constant Ki, determined by the double reciprocal plot was 0.08 μM, revealing a higher affinity of Sso1273 for α-chymotrypsin with respect to the cognate PEBP serine protease inhibitors.

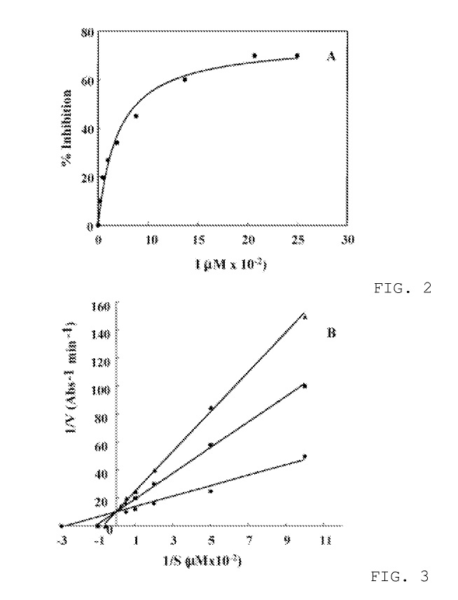

FIG. 3 shows the inhibition kinetics analysis. α-chymotrypsin (0.12 μM) was incubated, without (•) or with Sso0767 at 0.1 μM and 0.2 μM concentrations and assayed at increasing substrate concentrations. The reciprocals of the rate of the substrate hydrolysis for each inhibitor concentration were plotted against the reciprocals of the substrate concentrations. Ki was determined from the formula as per the competitive type of inhibition.

The Lineweaver-Burk reciprocal plot showed that Sso1273 was a competitive inhibitor for α-chymotrypsin. The Sso1273 IC50 value versus elastase was 0.15 μM (Table II), while the inhibition constant Ki determined using the double reciprocal plot was 0.10 μM (Table II). On the basis of these results, it can be claimed that Sso1273 substrate specificity reflects that of the I51 family typically inhibiting a-chymotrypsin, but not trypsin. In Table II are reported Ki and IC50 values that are the average of 3-5 separate determinations.

A careful study of the 3D model of Sso0767, taking into account the general structural implications needed for the interaction of the inhibitor with alpha-chymotrypsin and elastase, allowed identifying the aminoacidic region responsible for interacting with the target proteases. This sequence includes the structural motif T123-N130, in which the P1-P1′ site occurs between the residues L126 and E127. The T123-N130 sequence (TILLEIKN) (SEQ ID No. 67) is located in an external loop possessing an extended conformation, as observed in other protein inhibitors of proteases [Palmieri G, Catara G, Saviano M, Langella E, Gogliettino M, Rossi M. J “First Archaeal PEPB-Serine Protease Inhibitor from Sulfolobus solfataricus with Noncanonical Amino Acid Sequence in the Reactive-Site Loop”, Proteome Res. 2009 January; 8(1):327-34]. All these suggestions were confirmed by the design of several specific point mutations of the sequence inhibitor, including the replacement of the residue L126 (P1) with alanine or serine. Purified mutant inhibitors showed no ability to inhibit the target enzymes, although an analysis of secondary structure did not reveal any detectable conformational changes. Table III shows the sequence of the reactive site loop of the S. solfataricus inhibitor Sso0767 in comparison to that of several serine protease inhibitors. The results of these studies revealed that the inhibitor Sso0767 contains a new sequence in the protease binding site, that cannot be classified among the canonical consensus sequences of all serine protease inhibitors so far characterized. This unique motif may provide new insights into the inhibitor/protease binding mode.

TABLE II
kinetic parameters of Sso1273 versus the target proteases
bovine a-chymotrypsin and porcine Elastase

| Protease | IC₅₀ (μM) | Ki (μM) |
|---|---|---|
| Bovine a-chymotrypsin | 2.5 × 10⁻² | 8.0 × 10⁻² |
| porcine elastase | 4.0 × 10⁻² | 1.0 × 10⁻¹ |

TABLE III
The four conserved motifs found within the reactive
site loops (P3-P3′) of several serine protease
inhibitors are compared with that of SsCEI*

| Motif | P3 | P2 | P1 | P′1 | P′2 | P′3 |
|---|---|---|---|---|---|---|
| 1: Cys(P3)-Pro(P2) | Cys | Pro | Aaa | Aaa | Aaa | Aaa |
| 2: Thr(P2) | Aaa | Thr | Aaa | Aaa | Aaa | Aaa |
| 3: Cys(P2) | Aaa | Cys | Aaa | Aaa | Aaa | Aa |
| 4: Pro(P3) | Pro | Aaa | Aaa | Aaa | Aaa | Aaa |
| Sso0767 | Ile | Leu | Leu | Glu | Ile | Lys |

*The four conserved motifs (1-4) are indicated with the canonical residue/s found in the reactive site loop as previously reported (Apostoluk, et al, 1998). Each of the conserved sequence motif is representative of hundreds of serine protease inhibitors belonging to different families (Apostoluk, et al, 1998). Aaa denotes any amino acid.

### Example 6

### Inhibition of Human Acylamino-Acid-Releasing Enzyme (Named AARE or APEH) by the S. solfataricus Inhibitor Sso0767

The binding of Sso0767 to human acylamino-acid-releasing enzyme is shown in FIG. 4 where the hyperbolic curve indicates the best fit for the percentage inhibition data obtained, and the IC50 value was calculated from the graph.

Inhibitor effectiveness was determined by measuring enzyme activity both in the presence and absence of inhibitor. The activity of human acylamino-acid-releasing enzyme (AARE or APEH) (Takara) was measured by using the chromogenic substrate N-acetyl-Ala-pNA (Bachem) (40 μM in DMSO 100%). Stock solutions of lyophilized enzyme (0.5 uM) were prepared in distilled water and stored at −20° C. Hydrolysis of the substrate was performed at 37° C. for 15 min in 25 mM Tris-HCl buffer, pH 7.5. The reaction was stopped chilling the mixture in ice. Substrate hydrolysis was followed by recording the absorbance at 410 nm against a blank on a Hewlett-Packard spectrophotometer. The rate of increase in absorbance is a measure of the enzymatic activity and the decrease in this rate relative to a suitable control is used to ascertain inhibition. One unit of AARE activity (U) is defined as the amount of enzyme required to hydrolyze 1 nmol of substrate per min under the conditions of the assay using ε₄₁₀=8800 M⁻¹ cm¹.

The IC₅₀ value of the S. solfataricus inhibitor was determined by incubating increasing concentrations of the inhibitor with the enzyme AARE for 30 min at 37° C. in 25 mM Tris-HCl buffer, pH 7.5. Then, the substrate was added to the incubation mixture and the hydrolysis was performed for 30 min at 37° C. The data were fitted with the GraphPad Prism software. This analysis allowed the determination of IC50 values, which represent the concentrations at which the inhibition reached 50%. The inhibition followed a hyperbolic pattern with increasing concentrations of the inhibitor and IC₅₀ value at 37° C. was 20 nM.

### Example 7

### Inhibition of Human Acylamino-Acid-Releasing Enzyme (AARE) by Inhibitor SsCEI 1-4

Inhibition activity of the SsCEI 3 versus AARE (0.2 μg is shown in FIG. 5 wherein the protease was incubated with different compound concentrations and the residual protease activity was measured.

Inhibitor effectiveness of the present invention was determined by measuring enzyme activity both in the presence and absence of inhibitor SsCEI 1-4 using in vitro assay.

Velocities were fit to the equation for competitive inhibition for individual reactions of inhibitors with the enzyme using.

The SsCEI 1 to 4 reported in the Example 1 were tested for ability to inhibit the activity of human acylamino-acid-releasing enzyme. Stock solutions (1 mM) of each lyophilized compounds were prepared in DMSO 100% and stored at 4° C. Inhibition effectiveness of each compound was determined by measuring enzyme activity both in the presence and absence of compounds. The activity of human acylamino-acid-releasing enzyme (AARE) was measured by using the chromogenic substrate N-acetyl-Ala-pNA (Bachem) (40 μM in DMSO 100%) as described above. The IC50 values of the S. solfataricus compounds were determined by incubating increasing concentrations of the each compound with the enzyme AARE for 20 min at 37° C. in 25 mM Tris-HCl buffer, pH 7.5. Then, the substrate was added to the incubation mixture and the hydrolysis was performed for 20 min at 37° C. (FIG. 5). The data were fitted with the GraphPad Prism software. This analysis allowed the determination of IC50 values, which represent the concentrations at which the inhibition reached 50%. Using this methodology SsCEI 3 and SsCEI 4 of the present invention were found to exhibit inhibition activity towards human AARE protease. The IC50 values (See Table IV), obtained as average of 3-5 separate determinations, confirmed the utility of the compounds +of the present invention as effective AARE inhibitors.

TABLE IV
IC₅₀ values relative to SsCEI 1-4 towards
human acylamino-acid-releasing enzyme (AARE or
APEH).

| | IC₅₀ |
|---|---|
| PEPTIDIC INHIBITOR | (μM) |
| Compound 1 or SsCEI 1 | 0 |
| YAIDTILLEIKNINAD | |
| (SEQ ID No. 68) | |
| Compound 2 or SsCEI 2 | 0 |
| TILLEIKNINAD | |
| (SEQ ID No. 69) | |
| Compound 3 or SsCEI 3 | 25 |
| YAIDTILAEIKNINAD | |
| (SEQ ID No. 70) | |
| Compound 4 or SsCEI 4 | 35 |
| TILAEIKNINAD | |
| (SEQ ID No. 71) | |

### Example 8

### Inhibition of Porcine Acylamino-Acid-Releasing Enzyme (AARE) by Inhibitor SsCEI 1-4

The experiment described in example 7 has been performed using the porcine acylamino-acid-releasing enzyme. Both SsCEI 3 and SsCEI 4 have reduced the porcine APEH activity, although to different extents, with IC50 values of 20±0.4 μM and 35±0.8 μM respectively which were identical to those obtained using human APEH. The high affinity of SsCEI 4 towards porcine APEH was revealed by a Ki of 4.0±0.8 μM, as determined by the Lineweaver-Burk plot, showing also that SsCEI 4 is a competitive inhibitor of the porcine APEH (FIG. 7 A-B). The efficiency of SsCEI 4 can be ascribed to the APEH preference for an alanine residue, with respect to the leucine, at the P1 site, assuming that the SsCEI-APEH association occurs in a substrate-like manner

TABLE V
IC₅₀ values relative to SsCEI 1-4 towards
porcine acylamino-acid-releasing enzyme (AARE or
APEH).

| | IC₅₀ | Ki (μM) |
|---|---|---|
| PEPTIDIC INHIBITOR | (μM) | APEH |
| Compound 1 or SsCEI 1 | 0 | |
| YAIDTILLEIKNINAD | | |
| (SEQ ID No. 68) | | |
| Compound 2 or SsCEI 2 | 0 | |
| TILLEIKNINAD | | |
| (SEQ ID No. 69) | | |
| Compound 3 or SsCEI 3 SEQ | 25 | |
| YAIDTILAEIKNINAD | | 20 ± 0.4 |
| (SEQ ID No. 70) | | |
| Compound 4 or SsCEI 4 | 35 | |
| TILAEIKNINAD | | 4.0 ± 0.8 |
| (SEQ ID No. 71) | | |

### Example 9

### Down-Regulation of Proteasome Activity

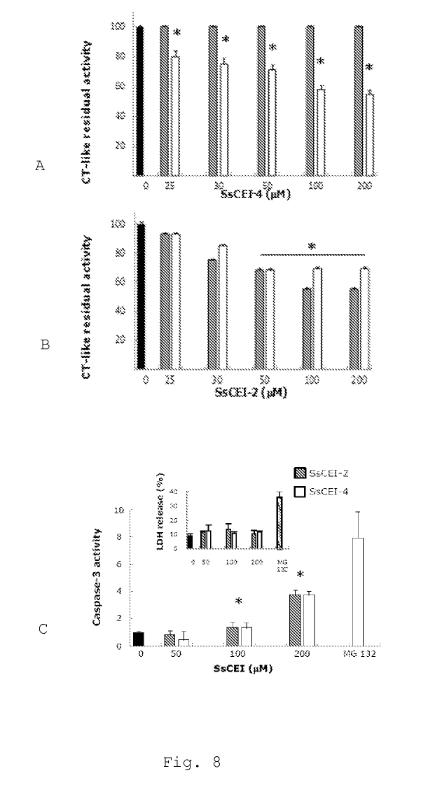

The proteasome down-regulation via APEH inhibition has been investigated. On the basis of the hypothesis that APEH can be used as a target to indirectly control/modulate proteasome functions in tumoral cells, an in vitro approach using the SsCEI 2, SsCEI 3 and SsCEI 4 peptides in the Caco-2 (human colon carcinoma) cell line has been carried out. To this end, differentiated Caco-2 cells were treated with peptides or with a specific proteasome inhibitor (MG132), for 48 h. As shown in FIGS. 8 A and B, SsCEI 2 and SsCEI 4 markedly reduced proteasomal activity. The results were compared to those obtained in the cell-free assays, where a purified proteasome fraction was used. Exposure to SsCEI 4 produced dose-dependent decrease (up to 45% of the residual activity) in the proteasome and APEH activity with respect to the untreated cultures, although this compound did not affect proteasome activity in cell-free systems, confirming that the proteasome is not the primary target for this inhibitor peptide. Instead, SsCEI 2 treatment led to down-regulation of proteasome in Caco-2 cells which was in agreement with the inhibitory effects shown by SsCEI in cell-free assays, demonstrating its ability to directly interact with the proteasome. In summary, SsCEI 2 and SsCEI 4 revealed to be direct or indirect modulator of the proteasome activity, while SsCEI 3 was unable to affect proteasome in Caco-2 cell.

To elucidate the molecular mechanisms associated with the inhibition of the proteasome, the effects of treatments with the SsCEI 2 and SsCEI 4 peptides on the activation of caspases have been evaluated. As shown in FIG. 8 C , caspase-3 activity, the key effector of apoptosis, was increased in a dose dependent manner. This was not associated with any cytotoxic effects, even at the highest concentration used (200 μM), as indicated by the lactate dehydrogenase (LDH) activity levels in the cell-culture media that remained comparable to the controls. Similar results were obtained testing SsCEI 2 and SsCEI 4 peptides on other tumoral cell lines such as MCF-7 (human breast adenocarcinoma) and U-87 (human glioblastoma astrocytoma, epithelial-like).

Therefore the preliminary hypothesis, supported by the provided experimental data, indicates that APEH is a valuable target for proteasome down-regulation.

### REFERENCES

Apostoluk, W.; Otlewski, J. (1998) Variability of the Canonical Loop Conformations in Serine Proteinases Inhibitors and Other Proteins. Proteins: Structure, Function, and Genetics, 32, 459-474.
Bruun A W, Svendsen I, Sorensen S O, Kielland-Brandt M C, Winther J R (1998) A high-affinity inhibitor of yeast carboxypeptidase Y is encoded by TFS1 and shows homology to a family of lipid binding proteins. Biochemistry 37:3351-3357.
Chen Q, Wang S, Thompson S N, Halland E D, Guttmann R P (2006) Identification and characterization of PEBP as a calpain substrate. J Neurochem 99:1133-1141.
Chong P K, Wright P C (2005) Identification and characterization of the Sulfolobus solfataricus P2 proteome. J Proteome Res 4:1789-1798
Hengst U, Albrecht H, Hess D, Monard D (2001) The phosphatidyletanolamine-binding protein is the prototype of a novel family of serine protease inhibitors. J Biol Chem 276:535-540.
Mima J, Narita Y, Chiba H, Hayashi R (2003) The multiple site binding of carboxypeptidase Y inhibitor (IC) to the cognate proteinase. Implications for the biological roles of the phosphatidylethanolamine-binding protein. J Biol Chem 278:29792-29800.
Serre L, Vallée B, Bureaud N, Schoentgen F, Zelwer C (1998) Crystal structure of the phosphatidylethanolamine-binding protein from bovine brain: a novel structural class of phospholipid-binding proteins. Structure 6:1255-1265.
Serre, L.; de Jesus, K. P.; Zelwer, C.; Bureaud, N.; Schoentgen, F.; Benedetti, H. (2001) Crystal structures of YBHB and YBCL from Escherichia coli, two bacterial homologues to a Raf kinase inhibitor protein. J Mol Biol., 310, 617-634
Yeung K, Seitz T, Li S, Janosch P, McFerran B, Kaiser C, Fee F, Katsanakis K D, Rose D W, Mischak H, Sedivy J M, Koich W (1999) Suppression of Raf-1 kinase activity and MAP kinase signalling by RKIP. Nature 401: 173-177.
Rawlings N. D., Tolle P. D., Barret J. A. (2004) Evolutionary families of peptidase inhibitors. Biochem. J. 378: 705-716

1-25. (canceled)

26. A proteic inhibitor belonging to PEBP family and characterised by the fact to be identified in and isolated from a hyper-thermophilic microorganism, wherein said hyper-thermophilic microorganism is Sulfolobus solfataricus, and to have the following characteristics:
a. molecular weight from 16 to 19 kDa and a pI value equal to 6.7
b. high heat stability and resistant to common denaturating agents of proteins, 100% of residual inhibitory activity after 4 days of incubation at 90° C.
c. resistant to proteolytic digestion by the common proteases.

27. A proteic inhibitor belonging to PEBP family with amino acid sequence of SEQ ID N.: 2.

28. A proteic inhibitor belonging to PEBP family enconded by the nucleic acid of SEQ ID N.: 1.

29. An isolated nucleic acid comprising the nucleic acid of SEQ ID N.:1 or variants thereof, where in the variants have one or more substitutions, insertions and/or deletions as compared to the nucleic acid of SEQ ID N.: 1 and encoded a protein characterized by high heat stability, resistance to common denaturating agents of proteins and proteolytic digestion by the common proteases and isolated from extremophilic microorganism, provided that:
a) these variants hybridize with a nucleic acid according to the sequence of SEQ ID N.:1 under stringent conditions, and further provided that these variants encode a protein having the same characteristics of proteic inhibitor of claim 26,
b) these variants have nucleic acid changes due to the degeneration of the genetic code and encoding the same amino acid having the same activity as the nucleic acid of SEQ ID N.: 1, and
c) the nucleic acid does not exclusively consist of the bases 1-471 of SEQ ID N.:1 or fragments thereof.

30. A nucleic acid which is a transcriptional product of one of the nucleic acids according to claim 29 or which selectively hybridises with such a transcription product under stringent conditions and is an antisense DNA or RNA.

31. An inhibitor active site sequence (T123-N130: TILLEIKN) (SEQ ID No. 67) that interacts with proteases and that represent a no canonic sequence no before known.

32. The manufacture of proteic inhibitor according to claim 26 by recombinant DNA technology in prokaryote and eukaryote or by chemical synthesis.

33. A vector which comprises a nucleic acid according to claim 29 and which is preferably a plasmid.

34. A peptide with formula I:
I: Y1-X1-X2-X3-X4-X5-X6-X7-X8-X9-X10-X11-X12-X13-
X14-X15-X16-Y2
wherein,
X1 is the L- or D-enantiomer of Tyrosine or null;
X2 is the L- or D-enantiomer of Alanine or null when X1 is null;
X3 is the L- or D-enantiomer of Isoleucine or null when X1 and X2 are null;
X4 is the L- or D-enantiomer of Aspartic acid or null when X1 through X3 are null;
X5 is the L- or D-enantiomer of Threonine or null when X1 through X4 are null;
X6 is the L- or D-enantiomer of Isoleucine, or D-enantiomer of Aspartic acid, or D-enantiomer of Cysteine with an acetamidomethyl (Acm) protection group on the sulphidryl group (abbreviated as Cys(Acm), or D-enantiomer of Proline;
X7 is the L- or D-enantiomer of Leucine or of Alanine, or the D-enantiomer of Methionine, or D-enantiomer of Cys(Acm), or D-enantiomer of Aspartic acid;
X8 is the L- or D-enantiomer of Leucine or of Alanine, or D-enantiomer of Methionine, or D-enantiomer of Cys(Acm);
X9 is the L- or D-enantiomer of Glutamic acid, or D-enantiomer of Aspartic acid or D-enantiomer of Cys(Acm), or D-enantiomer of Arginine, or D-enantiomer of Histidine;
X10 is the L- or D-enantiomer of Isoleucine or null when X11 through X16 are null;
X11 is the L- or D-enantiomer of Lysine or null when X12 through X16 are null;
X12 is the L- or D-enantiomer of Asparagine or null when X13 through X16 are null;
X13 is the L- or D-enantiomer of Isoleucine or null when X14 through X16 are null;
X14 is the L- or D-enantiomer of Asparagine or null when X15 and X16 are null;
X15 is the L- or D-enantiomer of Alanine or null when X16 is null; and
X16 is the L- or D-enantiomer of Aspartic acid or null.

35. Derivates of compound of formula I according to claim 34, where said derivates are selected form the group consisting of:
a) oligomers or multimers of molecules of the compound of formula I, said oligomers and multimers comprising two or more molecules of the compound of formula I each linked to a common scaffold moiety via an amide bond formed between an amine or carboxylic acid group present in molecules of the compound of formula I and an opposite amine or carboxylic acid group on a scaffold moiety said scaffold moiety participating in at least 2 amide bonds,
b) derivatives comprising a molecule of the compound of formula I or an oligomer or multimer as defined above in part a) conjugated via an ester bond, an ether bond or a thioether bond, on the N or C terminal amino- or carboxylic acid group of the peptide moiety to:
PEG,
PEG-based compounds,
fatty acids, and
salts and solvates of a molecule of the compound of formula I or of a derivative as defined in part a) or b) above.

36. Peptides generated by the general formula I according to claim 34, and carrying at least one methyl group on an amide nitrogen (N-methyl-aminoacids).

37. A host cell which has been transformed with a vector according to claim 33 and which is preferably an eukaryotic cell, more preferably a mammal cell, preferably an adult or embryonic stem cell, plant cell, yeast cell or insect cell, even more preferably a CHO, COS, HeLa, 293T, HEH o BHK, or a prokaryotic cell, preferably E. coli or Bacillus subtilis, wherein human embryonic stem cells are excluded.

38. A method to isolate gene of SEQ ID N. 1 according to claim 28 from total genomic DNA of hyperthermophilic microorganism S. solfataricus by polymerase chain reaction technique (PCR) using the following oligonucleotides:
(SEQ ID NO. 65)
P1 5′-CCATGGGCTTGAATAGTGAAAGTATATA-3′ [[e]]
and
(SEQ ID NO. 66)
P2 5′-GAGCTCTTTTCTCTTATATTTACCCATTACGAAT-3′.

39. A method of treating pathologies characterized by pathologic alteration of proteases activities, comprising administering to a subject in need thereof an effective amount of the proteic inhibitor according to claim 26.

40. A pharmaceutical composition comprising a therapeutically effective of a nucleic acid according to claim 28 in combination with a pharmaceutically acceptable carrier.

41. A pharmaceutical composition comprising a therapeutically effective dose of protein according to claim 26 in combination with a pharmaceutically acceptable carrier.

42. A method of treating pathologies characterized by pathologic alteration of proteases activities comprising administering to a subject in need thereof, an effective amount of the pharmaceutical composition according to claim 40.

43. The method according to claim 42, wherein the proteases are Serin-Protease.

44. The method according to claim 42, wherein said pathologies are Alzheimer's disease and pulmonary emphysema.

45. A method of treating pathologies characterized by alteration of proteasome proteolytic activity comprising administering to a subject in need thereof, an effective amount of the pharmaceutical composition according to claim 40.

46. A pharmaceutical composition comprising a therapeutically effective of a compound according to claim 34 in combination with a pharmaceutically acceptable carrier, for treating of cardiovascular diseases, cancer, inflammation, hematological diseases, neurological diseases and urological diseases.

47. A method to inhibit proteases keeping in touch with an appropriate amount of inhibitor according one of claim 26.

48. A method to inhibit the proteasome proteolytic components keeping in touch with an appropriate amount of inhibitor according to claim 26.

49. A method to inhibit proteases and regulate thereof activity when, in different pathologies, a proteolysis activity alteration is set (cancer, neuron-pathologies, pulmonary diseases e more over) keeping in touch with an appropriate amount of inhibitor according to claim 26.

50. A method of treating cardiovascular diseases, cancer, inflammation, hematological diseases, neurological diseases and urological diseases administering a therapeutically effective amount of a compound according to claim 26.


SEQUENCE LISTING (71 sequences)

SEQ ID NO: 1 - DNA, 474 bp, Sulfolobus solfataricus
ttgaatagtgaaagtatatattcgatgagagtagtatcttcagcctttaagaatgaggattttatacctattaaatatacttgtgatggacaagatctgtctccggagttagagtgggatcttgttactaacgctaaaagttatgcgataatcgtagaagatccagatgcgcctggaggaactttcatacattgggtaatatacaacataactaccaatagattgccagaaggagtgccaagactgtacaaatcacaatatggtgtacaaggtgtaaacgattttgggaatatagggtataacggtccgtgtcctcctaagacacatccacctcatagatattatttttatgtttatgcaattgatacaatactacttgaaattaaaaatattaatgctgataagttaaaatcactaatggagggacatgggatagagagaggattcgtaatgggtaaatataagagaaaataa

SEQ ID NO: 2 - protein, 157 aa, Sulfolobus solfataricus
MNSESIYSMRVVSSAFKNEDFIPIKYTCDGQDLSPELEWDLVTNAKSYAIIVEDPDAPGGTFIHWVIYNITTNRLPEGVPRLYKSQYGVQGVNDFGNIGYNGPCPPKTHPPHRYYFYVYAIDTILLEIKNINADKLKSLMEGHGIERGFVMGKYKRK

SEQ ID NO: 3 - protein, 4 aa, Sulfolobus solfataricus
ILLE

SEQ ID NO: 4 - protein, 4 aa, Sulfolobus solfataricus
ILAE

SEQ ID NO: 5 - protein, 4 aa, Sulfolobus solfataricus
ILLE

SEQ ID NO: 6 - protein, 4 aa, Sulfolobus solfataricus
ILAE

SEQ ID NO: 7 - protein, 4 aa, Sulfolobus solfataricus
ILLE

SEQ ID NO: 8 - protein, 4 aa, Sulfolobus solfataricus
ILLE

SEQ ID NO: 9 - protein, 4 aa, Sulfolobus solfataricus
ILLE

SEQ ID NO: 10 - protein, 4 aa, Sulfolobus solfataricus
ILLE

SEQ ID NO: 11 - protein, 4 aa, Sulfolobus solfataricus
ILAE

SEQ ID NO: 12 - protein, 4 aa, Sulfolobus solfataricus
ILAE

SEQ ID NO: 13 - protein, 4 aa, Sulfolobus solfataricus
ILAE

SEQ ID NO: 14 - protein, 4 aa, Sulfolobus solfataricus
ILAE

SEQ ID NO: 15 - protein, 4 aa, Sulfolobus solfataricus
ILLE

SEQ ID NO: 16 - protein, 4 aa, Sulfolobus solfataricus
ILLE

SEQ ID NO: 17 - protein, 4 aa, Sulfolobus solfataricus
ILLE

SEQ ID NO: 18 - protein, 4 aa, Sulfolobus solfataricus
ILLE

SEQ ID NO: 19 - protein, 4 aa, Sulfolobus solfataricus
ILLE

SEQ ID NO: 20 - protein, 4 aa, Sulfolobus solfataricus
ILLE

SEQ ID NO: 21 - protein, 4 aa, Sulfolobus solfataricus
ILLE

SEQ ID NO: 22 - protein, 4 aa, Sulfolobus solfataricus
ILAE

SEQ ID NO: 23 - protein, 4 aa, Sulfolobus solfataricus
ILAE

SEQ ID NO: 24 - protein, 4 aa, Sulfolobus solfataricus
ILAE

SEQ ID NO: 25 - protein, 4 aa, Sulfolobus solfataricus
ILAE

SEQ ID NO: 26 - protein, 4 aa, Sulfolobus solfataricus
ILAE

SEQ ID NO: 27 - protein, 4 aa, Sulfolobus solfataricus
ILAE

SEQ ID NO: 28 - protein, 4 aa, Sulfolobus solfataricus
ILAE

SEQ ID NO: 29 - protein, 4 aa, Sulfolobus solfataricus
ELLI

SEQ ID NO: 30 - protein, 4 aa, Sulfolobus solfataricus
EALI

SEQ ID NO: 31 - protein, 4 aa, Sulfolobus solfataricus
ELLI

SEQ ID NO: 32 - protein, 4 aa, Sulfolobus solfataricus
ELLI

SEQ ID NO: 33 - protein, 4 aa, Sulfolobus solfataricus
ELLI

SEQ ID NO: 34 - protein, 4 aa, Sulfolobus solfataricus
ELLI

SEQ ID NO: 35 - protein, 4 aa, Sulfolobus solfataricus
EALI

SEQ ID NO: 36 - protein, 4 aa, Sulfolobus solfataricus
EALI

SEQ ID NO: 37 - protein, 4 aa, Sulfolobus solfataricus
EALI

SEQ ID NO: 38 - protein, 4 aa, Sulfolobus solfataricus
EALI

SEQ ID NO: 39 - protein, 4 aa, Sulfolobus solfataricus
ELLI

SEQ ID NO: 40 - protein, 4 aa, Sulfolobus solfataricus
ELLI

SEQ ID NO: 41 - protein, 4 aa, Sulfolobus solfataricus
ELLI

SEQ ID NO: 42 - protein, 4 aa, Sulfolobus solfataricus
ELLI

SEQ ID NO: 43 - protein, 4 aa, Sulfolobus solfataricus
ELLI

SEQ ID NO: 44 - protein, 4 aa, Sulfolobus solfataricus
ELLI

SEQ ID NO: 45 - protein, 4 aa, Sulfolobus solfataricus
ELLI

SEQ ID NO: 46 - protein, 4 aa, Sulfolobus solfataricus
EALI

SEQ ID NO: 47 - protein, 4 aa, Sulfolobus solfataricus
EALI

SEQ ID NO: 48 - protein, 4 aa, Sulfolobus solfataricus
EALI

SEQ ID NO: 49 - protein, 4 aa, Sulfolobus solfataricus
EALI

SEQ ID NO: 50 - protein, 4 aa, Sulfolobus solfataricus
EALI

SEQ ID NO: 51 - protein, 4 aa, Sulfolobus solfataricus
EALI

SEQ ID NO: 52 - protein, 4 aa, Sulfolobus solfataricus
EALI

SEQ ID NO: 53 - protein, 16 aa, Sulfolobus solfataricus
YAIDTILLELKNINAD

SEQ ID NO: 54 - protein, 16 aa, Sulfolobus solfataricus
YAIDTILAEIKNINAD

SEQ ID NO: 55 - protein, 12 aa, Sulfolobus solfataricus
TILLEIKNINAD

SEQ ID NO: 56 - protein, 12 aa, Sulfolobus solfataricus
TILAEIKNINAD

SEQ ID NO: 57 - protein, 16 aa, Sulfolobus solfataricus
YAIDTILLEIKNINAD

SEQ ID NO: 58 - protein, 16 aa, Sulfolobus solfataricus
YAIDTILAEIKNINAD

SEQ ID NO: 59 - protein, 12 aa, Sulfolobus solfataricus
TILLEIKNINAD

SEQ ID NO: 60 - protein, 12 aa, Sulfolobus solfataricus
TILAEIKNINAD

SEQ ID NO: 61 - protein, 16 aa, Sulfolobus solfataricus
DANINKIELLITDIAY

SEQ ID NO: 62 - protein, 16 aa, Sulfolobus solfataricus
DANINKIEALITDIAY

SEQ ID NO: 63 - protein, 12 aa, Sulfolobus solfataricus
DANINKIELLIT

SEQ ID NO: 64 - protein, 13 aa, Sulfolobus solfataricus
DANINKIEALIYT

SEQ ID NO: 65 - DNA, 28 bp, Sulfolobus solfataricus
ccatgggcttgaatagtgaaagtatata

SEQ ID NO: 66 - DNA, 34 bp, Sulfolobus solfataricus
gagctcttttctcttatatttacccattacgaat

SEQ ID NO: 67 - protein, 8 aa, Sulfolobus solfataricus
TILLEIKN

SEQ ID NO: 68 - protein, 16 aa, Sulfolobus solfataricus
YAIDTILLEIKNINAD

SEQ ID NO: 69 - protein, 12 aa, Sulfolobus solfataricus
TILLEIKNINAD

SEQ ID NO: 70 - protein, 16 aa, Sulfolobus solfataricus
YAIDTILAEIKNINAD

SEQ ID NO: 71 - protein, 12 aa, Sulfolobus solfataricus
TILAEIKNINAD